(19) United States
(12) Patent Application Publication
(10) Pub. No.: US 2009/0198108 A1
(43) Pub. Date: Aug. 6, 2009

(54) Cervix Examination Device and Cervix Examination Set

(76) Inventors: Tien-Sheng Chen, Taipei City (TW); Hui-Bih Yuan, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/325,306

(22) Filed: Dec. 1, 2008

(30) Foreign Application Priority Data
Jan. 31, 2008 (TW) 097103790

(51) Int. Cl. A61B 1/32

(52) U.S. Cl. 600/220; 600/222

(57) ABSTRACT

This invention relates to a cervix examination device capable of containing and separating a light module. The cervix examination device comprises a first blade having a first open part and a first holding part; a second blade, which is pivoted to the first blade, comprising a second open part and a second holding part; an adjuster installed between the first and the second blades for adjusting the relative angle between the first and the second open parts; and a container installed inside the first open part, the container comprising an open portion and a closed portion, the light module can be contained in the closed portion via the open portion.

### BACKGROUND OF THE INVENTION

1. Field of the Invention

The present invention relates to a cervix examination device capable of containing and separating a light module; more particularly, the present invention relates to a cervix examination device suitable for cervical colposcopy to enable a user to distinguish abnormal cells from normal cells.

2. Description of the Related Art

Conventionally, diagnosis of cervical cancer performed with the cervical smear test, a technique known to have a rather high false negative rate. In order to prevent the above erroneous diagnosis, a popular technique called PAPSURE™, which provides a more accurate and efficient way, is often used in conjunction with the cervical smear test. The accuracy can be increased to more than 90% by the combination of the above two tests.

In order to conduct PAPSURE™, a vaginal speculum needs to be inserted into the vagina to facilitate inspection or medication. In addition, during such an examination, a light source is needed to illuminate the inside of the vagina. Generally, the light source is usually a handheld light projector or a head-wearable light. However, the traditional light source is not convenient and cannot be inserted inside the vagina to provide sufficient illumination; thus, the illumination effect is poor when such traditional light sources are used.

To provide more desirable illumination means, several solutions have been proposed in the related art. For example, a light module is attached to a second open part of a vaginal speculum. Because the light module is placed in the front of the vaginal speculum, the light module can provide illumination with higher efficiency inside the vagina. However, to prevent cross infection, the vaginal speculum and the light module need to be dispensed after used. It is a waste of resources and may increase costs if the light module cannot be reused.

### SUMMARY OF THE INVENTION

It is therefore an object of the present invention to provide a cervix examination device capable of containing and separating a light module.

It is another object of the present invention to provide a cervix examination set suitable for cervical colposcopy to enable a user to distinguish abnormal cells from normal cells.

To achieve the first object mentioned above, the cervix examination device of the present invention comprises a first blade having a first open part and a first holding part; a second blade, which is pivoted to the first blade, comprising a second open part and a second holding part; an adjuster installed between the first and the second blades for adjusting the relative angle between the first and the second open parts; and a container installed inside the first open part, the container comprising an open portion and a closed portion, the light module can be contained in the closed portion via the open portion.

To achieve another object mentioned above, the cervix examination set of the present invention comprises a first blade having a first open part and a first holding part; a second blade, which is pivoted to the first blade, comprising a second open part and a second holding part; an adjuster installed between the first and the second blades for adjusting the relative angle between the first and the second open parts; a light module for providing illumination; and a container installed inside the first open part, the container comprising an open portion and a closed portion, the light module can be contained in the closed portion via the open portion.

Other objects, advantages, and novel features of the present invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1 is an illustration of one embodiment of a cervix examination device of the present invention.

FIG. 2 is a cross-section view of the cervix examination device of the present invention.

FIG. 3 is an illustration of one embodiment of a light module of the cervix examination device of the present invention.

FIG. 4 is an illustration of one embodiment of a cervix examination set of the present invention.

### DETAILED DESCRIPTION OF THE PREFERRED EMBODIMENT

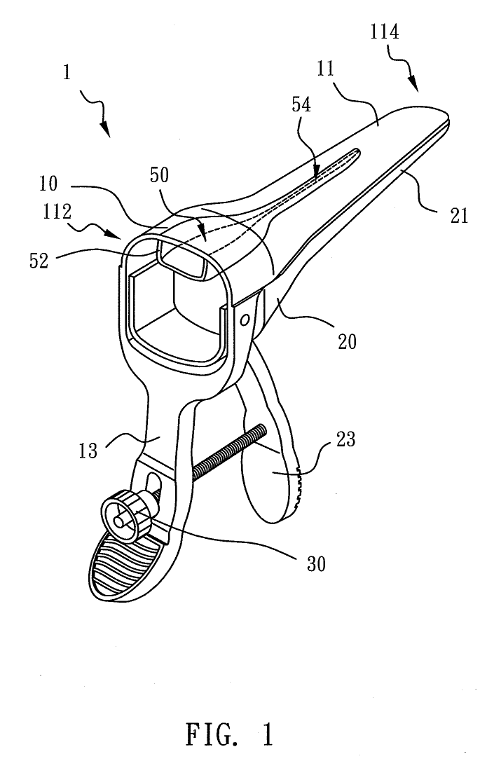

Please refer to FIG. 1 for an illustration of one embodiment of a cervix examination device of the present invention. The cervix examination device 1 of the present invention is suitable for cervical colposcopy to enable a user to distinguish abnormal cells from normal cells. The cervix examination device 1 mainly comprises a first blade 10, a second blade 20, an adjuster 30, and a container 50. The first blade comprises a first open part 11 and a first holding part 13. The second blade 20, which is pivoted to the first blade 10, comprises a second open part 21 and a second holding part 23. The adjuster 30 is installed between the first and the second blades 10, 20 for adjusting the relative angle between the first and the second open parts 11, 21. The container 50 comprises an open portion 52 and a closed portion 54. The container 50 is capable of containing and separating a light module. In the embodiment shown in FIG. 1, the container 50 is installed inside the first open part 11; however, the container 50 also can be installed inside the second open part 21, depending on the user's purpose.

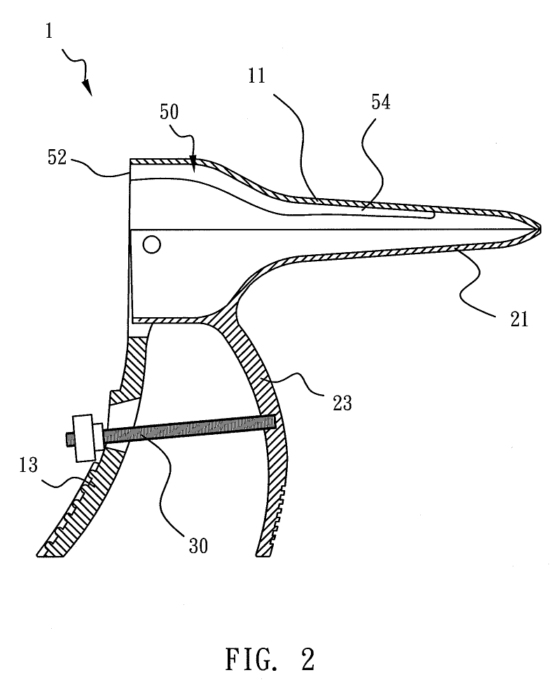

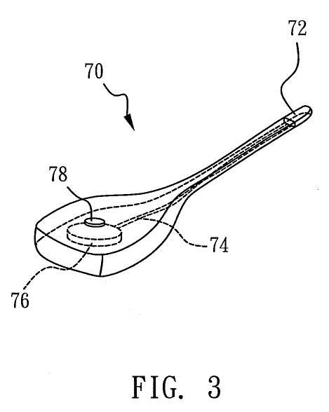

Please refer to FIG. 1 to FIG. 3. FIG. 2 is a cross-section view of the cervix examination device 1, and FIG. 3 is an illustration of a light module of the cervix examination device of the present invention. As shown in FIG. 1, the open portion of the container 50 is aligned to an end 112 of the first open part 11. The light module 70 can be isolated by the container 50 to prevent contact with the patient. The closed portion 54 of the container 50 is in a slender form and extends toward another end 114 of the first open part 11, thereby allowing the light module 70 to provide illumination more efficiently.

The whole light module 70 is contained and separated inside the container 50 to prevent contact with the patient; in other words, the light module 70 can be reused several times without causing cross infection. The light module 70 may comprise without limitation a light bulb and other electrical devices. The light module 70 also can be a fluorescent bar or other illumination objects. In the present embodiment, the light module 70 comprises a light unit 72, a control unit 76, and a switch 78. The light unit 72 is for emitting light. The control unit 76 is for controlling the light unit 72. The light unit 72 is electrically connected to the control unit 76 by a wire 74.

In most situations, the light unit 72 may emit light of a color different from that of the normal cells, preferably green light, which is complementary to the red light reflected by normal cells, so that users may distinguish abnormal cells from normal cells more easily. However, the light unit 72 can also emit other visible light with wavelengths smaller than those of red light, for example and without limitation white light, yellow light, blue light, purple light, etc.

The light unit 70 may comprise a single color or multi-color LED (light emitting diode), or comprise a plurality of LEDs of different colors. The control unit 76 controls on/off and even controls the color of the light emitted by the light unit 72 according to the switch 78, operated by the user. In the present embodiment, the light module 70 is powered by a battery.

In addition, in the present embodiment, the light module 70 has at least one part made of soft material in order to move the light unit 72 to the closed portion 54 easily. Furthermore, the switch 78 has a bump shape such that when the light module 70 is inserted completely into the container 50, the switch 78 will be pressed by the end 112 of the first open part 11, and therefore the light unit 72 will be turned on automatically.

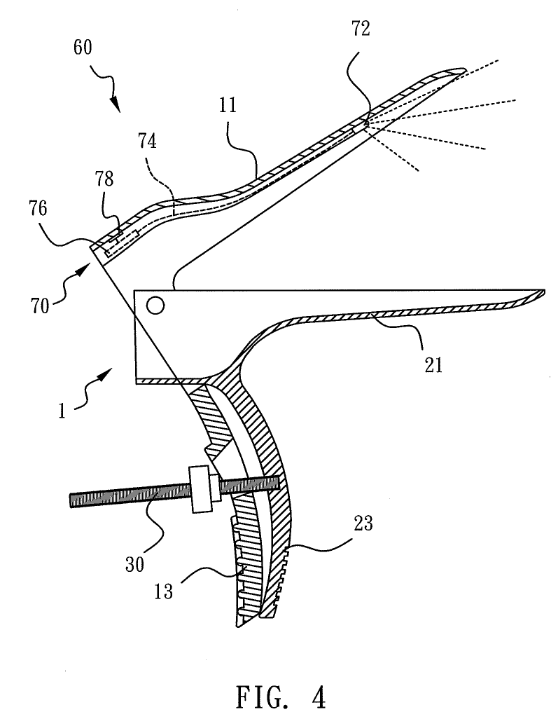

Please refer to FIG. 1 and FIG. 4. FIG. 4 is an illustration of one embodiment of a cervix examination set of the present invention. The cervix examination set 60 is a combination of the cervix examination device 1 and the light module 70. When the user operates the adjuster 30 to move the first holding part 13 closer to the second holding part 23, the relative angle between the first open part 11 and the second open part 21 will be increased. The light module 70 is accommodated in the container 50, which is installed inside the first open part 11. The light unit 72 is moved to the closed portion 54 with the assistance of the wire 74 in order to provide illumination with greater efficiency. The user can operate the switch 78 to switch the light on/off or change the color of the light emitted by the unit 72 according to his or her requirements. Because the light module 70 is isolated inside the container 50 during the inspection process, the light module 70 will not be contaminated by contact with the patient. Therefore, after the inspection is finished, the user only needs to dispense the cervix examination device 1, and the light module 70 can be easily detached from the container 50 and put into another cervix examination device for reuse.

The cervix examination set 60 of the present invention may be applied to both PAPSURE™ and ordinary cervical observations.

In contrast to the prior art, the cervix examination device 1 of the present invention is capable of containing and separating the light module 70 inside the container 50 to prevent the light module 70 from being contaminated, thus preventing cross infection. In addition, the light module 70 of the cervix examination set 60 can be a reusable resource, thus reducing the cost of the cervical examination.

Although the present invention has been explained in relation to its preferred embodiments, it is to be understood that many other possible modifications and variations can be made without departing from the spirit and scope of the invention as hereinafter claimed.

What is claimed is:

1. A cervix examination device capable of containing and separating a light module, the cervix examination device comprising:
a first blade having a first open part and a first holding part;
a second blade, which is pivoted to the first blade, comprising a second open part and a second holding part;
an adjuster installed between the first and the second blades for adjusting the relative angle between the first and the second open parts; and
a container installed inside the first open part, the container comprising an open portion and a closed portion; the light module can be contained in the closed portion via the open portion.

2. The cervix examination device as claimed in claim 1, wherein the open portion of the container is aligned to an end of the first open part.

3. The cervix examination device as claimed in claim 1, wherein the open portion of the container is recessed inside an end of the first open part.

4. The cervix examination device as claimed in claim 1, wherein the closed portion of the container is in a slender form.

5. A cervix examination set comprising:
a first blade having a first open part and a first holding part;
a second blade, which is pivoted to the first blade, comprising a second open part and a second holding part;
an adjuster installed between the first and the second blades for adjusting the relative angle between the first and the second open parts;
a light module for providing illumination; and
a container installed inside the first open part, the container comprising an open portion and a closed portion; the light module can be contained in the closed portion via the open portion.

6. The cervix examination set as claimed in claim 5, wherein the light module comprises:
a light unit for emitting light; and
a control unit electrically connected to the light unit for controlling the light unit.

7. The cervix examination set as claimed in claim 6, wherein the control unit controls the color of light emitted by the light unit.

8. The cervix examination set as claimed in claim 6, wherein the light unit is capable of emitting white light.

9. The cervix examination set as claimed in claim 6, wherein the light unit is capable of emitting green light.

10. The cervix examination set as claimed in claim 6, wherein the light unit is capable of emitting blue light.

11. The cervix examination set as claimed in claim 6, wherein the light unit is capable of emitting yellow light.

12. The cervix examination set as claimed in claim 6, wherein the light unit is capable of emitting purple light.

13. The cervix examination set as claimed in claim 6, wherein the light unit comprises a light emitting diode.

14. The cervix examination set as claimed in claim 6, wherein the light module at least has one part made of soft material.

15. The cervix examination set as claimed in claim 6, wherein the light module further comprises a switch for controlling the control unit.

16. The cervix examination set as claimed in claim 15, wherein the switch has a bump shape.

17. The cervix examination set as claimed in claim 5, wherein the open portion of the container is aligned to an end of the first open part.

18. The cervix examination set as claimed in claim 5, wherein the open portion of the container is recessed inside an end of the first open part.

19. The cervix examination set as claimed in claim 5, wherein the closed portion of the container is in a slender form.
